(19) United States
(12) Patent Application Publication
(10) Pub. No.: US 2025/0049038 A1
(43) Pub. Date: Feb. 13, 2025

(54) ANHYDROUS SILVER COMPOSITIONS AND USES THEREOF

(71) Applicant: AMERICAN SILVER, LLC, American Fork, UT (US)

(72) Inventors: Robert J. Holladay, Saratoga Springs, UT (US); Nathan R. Moeller, Highland, UT (US); Jerry S. Revelli, Highland, UT (US)

(21) Appl. No.: 18/720,433

(22) PCT Filed: Dec. 15, 2022
(86) PCT No.: PCT/US2022/053049

§ 371 Date: Jun. 14, 2024

Related U.S. Application Data
(60) Provisional application No. 63/290,032, filed on Dec. 15, 2021.

(51) Int. Cl. A01N 59/16; A01N 25/12; A01N 25/34; A01P 1/00

(52) U.S. Cl. CPC A01N 59/16; A01N 25/12; A01N 25/34; A01P 1/00

(57) ABSTRACT

Compositions that contain a substrate and silver particles are provided. where the composition are anhydrous, optionally having antimicrobial properties in either the anhydrous or a rehydrated form.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

The present invention relates generally to anhydrous silver compositions optionally having antimicrobial properties, and the manufacture and use of the compositions.

### BACKGROUND OF THE INVENTION

The effective and efficient delivery and use of anti-microbial agents is an area of active research. The present disclosure addresses this need by providing compositions that may be used to deliver and provide antimicrobial properties.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 shows a scanning electronic micrograph (SEM) of a powdered anhydrous composition of the present disclosure where silver particles are visible as bright spots.

FIG. 2 shows a SEM of a powdered anhydrous composition of the present disclosure where silver particles are visible as bright spots.

FIG. 3 shows a SEM of a fibrous anhydrous composition of the present disclosure where the silver particles are visible as bright spots.

### DETAILED DESCRIPTION OF THE INVENTION

The present invention may be understood more readily by reference to the following detailed description of preferred embodiments of the invention and the Examples included herein. In reading this detailed description, and unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In brief, the present disclosure provides anhydrous compositions that include colloidal silver particles, as well as methods of making and using the anhydrous compositions. In one embodiment, the anhydrous composition includes both a silver particle and a substrate. Optionally, the anhydrous composition may include additional components.

“Silver” as used herein refers to the metal silver (or “Ag”), which may be in any of a number of different forms (e.g., particles including particles with neutral silver metal, ions and salts, and mixtures thereof). The terms “colloidal silver”, “particulate silver” and “silver particles” are used interchangeably to refer to discrete particles that comprise silver. In one embodiment, colloidal silver refers to particles which in whole or in part comprise silver, where the particles are suspended in an aqueous medium. Representative examples of silver as utilized herein includes colloidal silver having a coating of silver oxide, e.g., Ag₂O or Ag₄O₄).

In one embodiment, the silver particle has a silver core surrounded by a multivalent silver oxide coating comprised of Ag₄O₄ molecules, i.e., having an interior of metallic silver and an exterior surface of silver oxide. In one embodiment the compositions contain silver in the +2 oxidation state, particularly in association with metallic silver, to provide potent antimicrobial properties. This high potency means that lower amounts of silver particles may be utilized in the preparation of the anhydrous compositions of the present disclosure, thus avoiding undesirable side-reactions that can occur with silver, e.g., discoloration of the composition, color instability, rheology instability, and oxidation of components of the composition. The silver particle may comprise a plurality of silver particles that may be characterized by particle size. In one embodiment, the silver particles range in size from 1-100 nanometers, while in one embodiment, the silver particles have an average diameter of between 1 nanometer and 100 nanometers. In one embodiment, more than 50% of the silver particles have a maximum dimension of less than 0.015 micrometers. In one embodiment, at least 75% of the silver particles have diameters between 0.005 micrometers and 0.015 micrometers.

The particles may optionally be characterized in terms of valence of the silver. In one embodiment, the silver particles include both silver in the zero-valent state represented as [Ag (0)], i.e., metallic silver, and a coating of silver oxide in an oxidation state selected from Ag(I), Ag(II), and Ag(III). For example, the particles may have a coating of silver oxide, e.g., Ag₂O or Ag₄O₄. In one embodiment, the silver oxide in the particles may be primarily in the form of Ag₄O₄, i.e., silver II oxide. In a molecule of this material two of the silver atoms are in the 1+ state (silver I) while the other two silver molecules are in the 3+ state (silver III). Under certain conditions these molecules can give rise to silver atoms or ions in the 2+ (silver II) state. Thus, the present disclosure provides silver particles comprising metallic silver and silver oxide, the silver oxide being selected from Ag₂O and Ag₄O₄.

The silver particle may be provided as an aqueous composition. For example, the silver particles may be provided as a suspension in water, e.g., a stable suspension, of colloidal silver, where the silver particle has a silver core surrounded by a multivalent silver oxide coating comprised of Ag₄O₄ molecules, i.e., having an interior of metallic silver and an exterior surface of silver oxide. In one embodiment the aqueous silver composition is colorless. The silver particles in the aqueous composition may be characterized by particle size as described elsewhere herein. As an aqueous composition, in one embodiment the silver particles are present in the composition at a concentration of 1 ppm to about 1,000 ppm.

In one embodiment, the particles contain a thin multivalent silver oxide coating, built of numerous Ag₄O₄ molecules which surround a metallic nano-sized silver core. With the Ag₄O₄ coating, the nano silver particle is attracted to the surrounding water molecules, and as such, becomes part of the structure of the water. This makes the silver much more stable and bioavailable than other forms of silver. Within optional embodiments of the invention these particles can range in size from 1-100 nanometers, or from 1-10 nanometers, or from 5-7 nanometers. Moreover, within preferred embodiments the particles are surrounded by a multivalent silver oxide coating comprised of Ag₄O₄ molecules.

In one embodiment, the anhydrous compositions of the present disclosure include ionic silver, however in another embodiment, the anhydrous compositions of the present disclosure do not include ionic silver, or else they contain minimal amounts (e.g., less than 5%, 2%, 1%, 0.1%, 0.01%) of ionic silver. Ionic silver may be omitted from anhydrous compositions of the present disclosure in order to avoid silver metabolism, which may lead to silver being bound up in the body and causing a skin condition known as Argyria, otherwise known as the blue man's syndrome.

Exemplary aqueous compositions comprising colloidal silver particles are described in, and may be prepared by techniques disclosed in, U.S. Pat. Nos. 6,214,299; 6,743,348; 7,135,195; and 8,535,728 as well as U.S Publication No. 2011/0262556. For example, the preparation of a composition comprising colloidal silver particles may utilize an electrochemical cell comprising electrodes. The process comprises the steps of: (a) placing a silver electrode in contact with a quantity of high purity water; (b) conveying electrical current through the silver electrode to thereby separate particles of silver from said silver electrode in a manner sufficient to cause production of suspended silver particles within the water; and (c) agitating the water during said production of suspended silver particles to thereby disperse the silver particles into a more uniform concentration within said water such that a higher quantity of colloidal silver particles can be produced per batch.

As another example, the preparation of an aqueous composition comprising colloidal silver particles may comprise the steps of: (a) establishing an electrical circuit comprising a current source, and a first conductor electrically connected to said current source and a second conductor electrically connected to said current source, wherein said first conductor is disposed spaced apart from said second conductor, and wherein at least one of the conductors is made of elemental silver; (b) closing the circuit by placing the first conductor and the second conductor in communication with a fluidic resistor; (c) operating the current source to supply alternating current simultaneously to the first conductor and the second conductor such that voltage is increasing and decreasing within the first and second conductors in alternating tandem to thereby cause silver particles to separate from the first electrode and enter the fluidic resistor and become disposed in suspension within said fluidic resistor; and (d) selectively adjusting the electrodes by moving them toward the fluidic resistor to compensate for decrease in electrode length due to gradual separation of silver particles therefrom to thereby prevent arcing from occurring between the electrodes and said fluidic resistor.

Other suitable aqueous colloidal silver particles and their preparation are described in, e.g., PCT Publication No. WO 2009/009143 and US Publication No. 2010/0187091.

In one embodiment, the colloidal silver particles are stable in essentially pure water without surfactants, etc. Additionally, or in another embodiment, the solution of colloidal silver particles is essentially colorless. In another embodiment, the colloidal silver particles are in saline. In another embodiment, the colloidal silver particles are in combination with electrolytes needed by a body, e.g., potassium and magnesium. In one embodiment, the colloidal silver particles are in saline, and the saline contains electrolytes.

The anhydrous compositions of the present disclosure include a substrate in addition to silver. In one embodiment the substrate is a solid. In one embodiment the anhydrous composition is a solid. In one embodiment the substrate and/or the anhydrous composition is in a powdered form. In one embodiment, the powdered form is a free-flowing powder. In one embodiment the anhydrous compositions of the present disclosure are in a loose powder form. In a powdered form, the anhydrous composition is not adhered to any solid surface. Thus, in one embodiment, the anhydrous compositions of the present disclosure are not a coating or a component in a coating which is adhered to a solid surface. In one embodiment the anhydrous compositions of the present disclosure are packaged in a jar or tube or bag, or other such container that allows for storage and transport of the anhydrous composition to an end-user. Thus, in one embodiment the present disclosure provides an anhydrous composition as disclosed herein within suitable packaging, e.g., a jar or bag. In one embodiment the present disclosure provides suitable packaging that contains only an anhydrous composition as disclosed herein in a powdered form, particularly a free-flowing or loose powder powdered form. In one embodiment the substrate and/or the anhydrous composition is fibrous. In one embodiment the substrate is a textile. In one embodiment the substrate is a paper product. In one embodiment the substrate is personal protective equipment, e.g., a mask or a glove.

In one embodiment the substrate is a food, or is a food additive, collectively a foodstuff. In one embodiment the food is sugar. In one embodiment the sugar is synthetic sugar. Exemplary synthetic sugars include, without limitation, allulose™, stevia™, splenda™, truvia™, swerve™, and purcane™ sweeteners. In one embodiment the sugar is a natural sugar. Exemplary natural sugars include, without limitation, a carbohydrate selected from cane sugar such as sucrose, beet sugar such as sucrose, dairy sugar such as lactose, and fruit sugar such as fructose. In one embodiment the substrate is a non-carbohydrate sweetener. Exemplary non-carbohydrate sweeteners include, without limitation, sorbitol and xylitol.

In one embodiment, the substrate is an inorganic material. Exemplary inorganic materials include, without limitation, aluminosilicate, inorganic oxide materials such as silicon oxide such as silicon dioxide including silica, and metal oxides such as aluminum oxide, zinc oxide and calcium oxide. Optionally, the metal oxide substrate does not include silver, i.e., the metal of the metal oxide substrate does not include silver.

In one embodiment, the substrate is a polymer, such as a non-edible polymer. In one embodiment the polymer is a synthetic polymer, where exemplary synthetic polymers include, without limitation, polyethylene and polypropylene.

As mentioned elsewhere herein, the present disclosure provides an anhydrous composition that includes both silver particle and a substrate. In one embodiment the silver particle is a coating on the substrate. For example, the silver particle is a coating of silver particles distributed, optionally uniformly or evenly distributed, on a surface or the entire surface of the substrate. In one embodiment the silver particle adheres to a surface of the substrate. In one embodiment the substrate is in a particulate form, e.g., a free-flowing particulate form. In one embodiment the substrate in the anhydrous composition is in a powdered form. In one embodiment the substrate in the anhydrous composition is in a fibrous form. In one embodiment the substrate may be either powdered or fibrous but is in a particulate form that provides for an anhydrous composition that is free flowing, e.g., may be poured from a jar or other container. Thus, in one embodiment when the substrate is in a fibrous form, the fibers are sufficiently small, e.g., in embodiments are under 1 inch or under 0.5 inches, or under 0.1 inches, that the composition can be poured from a container, or scooped from the container with a spoon or the like.

In one embodiment the anhydrous composition of the present disclosure has antimicrobial properties. In one embodiment the silver particle in the anhydrous composition is a plurality of silver particles such that the anhydrous composition demonstrates antimicrobial properties. In other words, the silver particles are present in the anhydrous composition at a concentration effective to demonstrate antimicrobial properties.

In one embodiment the anhydrous composition may be characterized by the weight percent of the substrate and/or the weight percent of the silver particles present in the anhydrous composition. In one embodiment a weight percent of the substrate is greater than a weight percent of the silver particles. In one embodiment a weight percent of the substrate is greater than 75% and a weight percent of the silver particles is less than 25%.

In one embodiment the anhydrous composition may be prepared by combining an aqueous composition comprising silver particle, and combining that composition with a substrate to provide an aqueous silver composition. The aqueous silver composition is then subjected to water-removing conditions such as evaporation. In one embodiment the aqueous silver composition is placed in a freeze dryer and the water is removed at lower temperature under reduced pressure, to provide the anhydrous composition. The result of the process may be an anhydrous composition in a powdered form, or in a fibrous form, as two examples. The powdered or fibrous form contains particulate substrate where the particles have a coating of silver particles, where the coating may be evenly or uniformly distributed on the surface of the particulate substrate.

The water is removed from the aqueous silver composition to provide an anhydrous composition. As used herein, an anhydrous composition contains little or no water, and is at least dry to the touch. In embodiments, the anhydrous composition contains less than 1 wt % water, or less than 0.1 wt % water, or less than 0.01 wt % water. Water content may be measured by conventional means, e.g., a Karl Fischer titration.

In one embodiment, the anhydrous powdered form is a free-flowing powder. In one embodiment the anhydrous powdered compositions of the present disclosure are in a loose powder form. In a powdered form, the anhydrous composition is not adhered to any solid surface. Thus, in one embodiment, the anhydrous compositions of the present disclosure are not a coating or a component in a coating which is adhered to a solid surface. In one embodiment the anhydrous compositions of the present disclosure are packaged in a jar or tube or bag, or other such container that allows for storage and transport of the powdered anhydrous composition to an end-user. Thus, in one embodiment the present disclosure provides an anhydrous composition as disclosed herein within suitable packaging, e.g., a jar or bag. In one embodiment the present disclosure provides suitable packaging that contains only an anhydrous composition as disclosed herein in a powdered form, particularly a free-flowing or loose powder powdered form. In one embodiment the substrate and/or the anhydrous composition is fibrous.

In one embodiment the anhydrous composition is rehydrated. Thus, the present disclosure provides a method of preparing an aqueous composition comprising combining an anhydrous composition as described herein with water to provide a rehydrated composition.

In one embodiment the present disclosure provides a method of imparting antimicrobial properties to a substrate, the method comprising: combining the substrate with a composition comprising silver particle and water to provide an aqueous composition; and removing water from the aqueous composition to provide an anhydrous composition comprising the silver particle and the substrate.

In one embodiment the present disclosure provides a method for providing preserved food, the method comprising: providing food, optionally providing solid food; combining the food with an aqueous composition comprising silver particles to provide a precursor composition; and removing water from the precursor composition to provide the preserved food. Optionally, the preserved food may be rehydrated by the addition of water.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y,” and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

The following Examples are offered by way of illustration and not by way of limitation. For example, synthetic silicon dioxide is an exemplary substrate. Other suitable substrates are described herein. For example, other inorganic materials may serve as the substrate, e.g., aluminosilicate, inorganic oxide materials such as silicon oxide such as silicon dioxide including silica, and metal oxides such as aluminum oxide, zinc oxide and calcium oxide.

### EXAMPLES

### Example 1

An aqueous suspension of colloidal silver particles (15 g, SilverSol™ technology, 1 ppm silver particles, American Biotech Labs, American Fork, UT) is combined with synthetic silicon dioxide (5 g, Flo-gard™ FF silica, PPG Industries, Pittsburgh, PA) with gentle mixing to provide an aqueous silver composition. The water is removed from the aqueous silver composition with a freeze dryer to provide an anhydrous composition where the silver particles are evenly distributed on the silica at a concentration of 3 ppm silver particles.

### Example 2

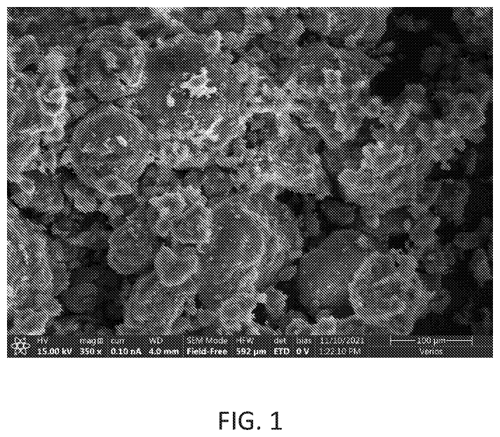

An aqueous suspension of colloidal silver particles (15 g, SilverSol™ technology, 60 ppm silver particles, American Biotech Labs, American Fork, UT) was combined with synthetic silicon dioxide (5 g, Flo-gard™ FF silica, PPG Industries, Pittsburgh, PA) with gentle mixing to provide an aqueous silver composition. The water was removed from the aqueous silver composition with a freeze dryer to provide a powdered anhydrous composition where the silver particles were evenly distributed on the silica at a concentration of 184 ppm silver particles. FIG. 1 provides a scanning electronic micrograph (SEM) of the anhydrous composition where the silver particles are visible at bright spots. Energy dispersive x-ray (EDX) analysis of the anhydrous composition confirmed the elemental identification and composition.

### Example 3

An aqueous suspension of colloidal silver particles (15 g, SilverSol™ technology, 500 ppm silver particles, American Biotech Labs, American Fork, UT) is combined with synthetic silicon dioxide (5 g, Flo-gard™ FF silica, PPG Industries, Pittsburgh, PA) with gentle mixing to provide an aqueous silver composition. The water is removed from the aqueous silver composition with a freeze dryer to provide an anhydrous composition where the silver particles are evenly distributed on the silica at a concentration of 1,533 ppm silver particles.

### Example 4

An aqueous suspension of colloidal silver particles (15 g, SilverSol™ technology, 1,000 ppm silver particles, American Biotech Labs, American Fork, UT) is combined with synthetic silicon dioxide (5 g, Flo-gard™ FF silica, PPG Industries, Pittsburgh, PA) with gentle mixing to provide an aqueous silver composition. The water is removed from the aqueous silver composition with a freeze dryer to provide an anhydrous composition where the silver particles are evenly distributed on the silica at a concentration of 3,067 ppm silver particles.

Example 5

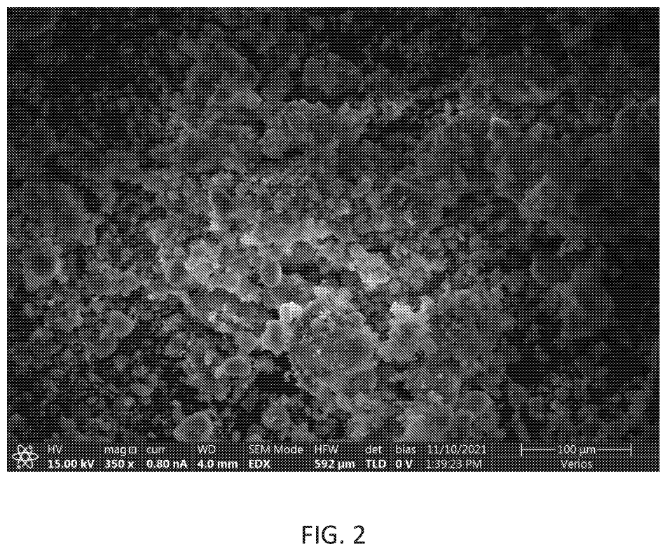

An aqueous suspension of colloidal silver particles (15 g, SilverSol™ technology, 60ppm silver particles, American Biotech Labs, American Fork, UT) was combined with synthetic silicon dioxide (5 g, Flo-gard™ LPC silica, PPG Industries, Pittsburgh, PA) with gentle mixing to provide an aqueous silver composition. The water was removed from the aqueous silver composition with a freeze dryer to provide a powdered anhydrous composition where the silver particles are evenly distributed on the silica at a concentration of 184 ppm silver particles. FIG. 2 provides a SEM of the anhydrous composition where the silver particles are visible at bright spots. EDX analysis of the anhydrous composition confirmed the elemental identification and composition.

### Example 6

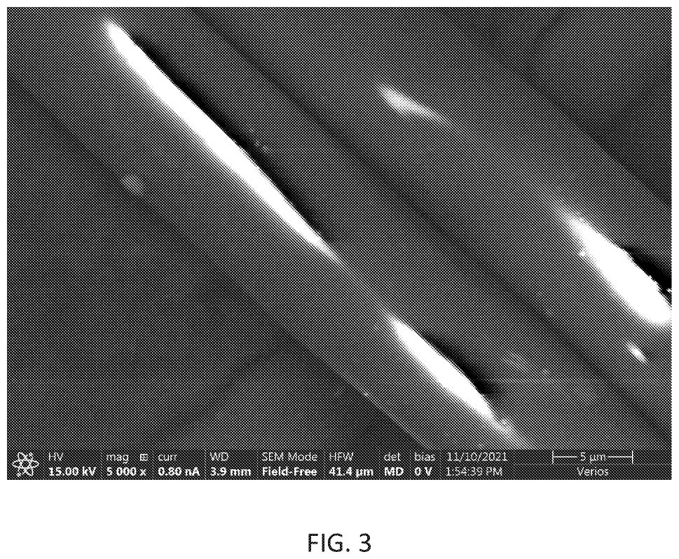

An aqueous suspension of colloidal silver particles (SilverSol™ technology, American Biotech Labs, American Fork, UT) was sprayed onto a medical pad (ASO Medical, Sarasota FL) to provide an aqueous silver composition. The water was removed from the aqueous silver composition by evaporation to provide a fibrous anhydrous composition where the silver particles are evenly distributed on the medical pad. FIG. 3 provides a SEM of the fiber sample where the silver particles are visible at bright spots. EDX analysis of the anhydrous composition confirmed the elemental identification and composition.

### Example 7

Compositions where prepared containing 5 g of powdered zinc oxide and 15 g of an aqueous composition containing 60 ppm of colloidal silver. These compositions were treated as described herein to remove water and thus provide a powdered anhydrous composition of the present disclosure. The zinc oxide-containing compositions were re-suspended and then evaluated for anti-microbial activity. Zone of Inhibition studies determined that the re-suspended zinc oxide compositions were effective anti-microbial agents against MRSA at a MRSA concentration of 2.0×10⁶, using 10 microliters of re-suspended zinc oxide composition at a concentration of 100 mg/ml of zinc oxide. At a zinc oxide concentration of 50 mg/ml and 100 mg/mL, the re-suspended zinc oxide composition was an effective anti-microbial agent against Psuedomonas aeruginosa 19660 (a zone of inhibition was observed). At each of a zinc oxide concentration of 10 mg/mL, 50 mg/ml and 100 mg/mL, the re-suspended zinc oxide composition created a zone of inhibition against Candida albicans 24433.

### SUMMARY OF THE INVENTION WITH EXEMPLARY EMBODIMENTS

All references disclosed herein, including patent references and non-patent references, are hereby incorporated by reference in their entirety as if each was incorporated individually.

In brief, the present disclosure provides anhydrous compositions that include colloidal silver particles, as well as methods of making and using the anhydrous compositions. In one embodiment, the anhydrous composition includes both a silver particle and a substrate. Optionally, the anhydrous composition may include additional components.

The following are exemplary embodiments of the present disclosure, numbered for convenience.

1. An anhydrous composition comprising a silver particle and a substrate.

2. The composition of embodiment 1 wherein the substrate is a synthetic sugar.

3. The composition of embodiment 2 wherein the synthetic sugar is selected from allulose, stevia, splenda, truvia, swerve, and purcane sweeteners.

4. The composition of embodiment 1 wherein the substrate is a natural sugar.

5. The composition of embodiment 4 wherein the natural sugar is a carbohydrate selected from cane sugar such as sucrose, beet sugar such as sucrose, dairy sugar such as lactose, and fruit sugar such as fructose.

6. The composition of embodiment 1 wherein the substrate is a non-carbohydrate sweetener.

7. The composition of embodiment 6 wherein the non-carbohydrate sweetener is selected from sorbitol and xylitol.

8. The composition of embodiment 1 wherein the substrate is an inorganic material, such as an inorganic material selected from aluminosilicate, silicon oxide such as silicon dioxide including silica, and metal oxides such as aluminum oxide, zinc oxide and calcium oxide.

9. The composition of embodiment 1 wherein the substrate is a polymer.

10. The composition of embodiment 9 wherein the polymer is a synthetic polymer.

11. The composition of embodiment 10 wherein the synthetic polymer is selected from polyethylene and polypropylene.

12. The composition of embodiments 1-11 wherein the substrate is a solid.

13. The composition of embodiments 1-12 wherein the composition is a solid.

14. The composition of embodiments 1-13 wherein the substrate is in a powdered form.

15. The composition of embodiments 1-13 in a powdered form.

16. The composition of embodiments 1-13 wherein the substrate is fibrous.

17. The composition of embodiments 1-15 wherein the substrate is selected from food and food additives.

18. The composition of embodiments 1-15 wherein the substrate is a textile.

19. The composition of embodiments 1-15 wherein the substrate is a paper product.

20. The composition of embodiments 1-15 wherein the substrate is personal protective equipment, e.g., a mask.

21. The composition of embodiments 1-20 wherein the silver particle is a coating on the substrate.

22. The composition of embodiments 1-21 wherein the silver particle is a coating of silver particles evenly distributed on a surface of the substrate.

23. The composition of embodiments 1-22 wherein the silver particle adheres to the substrate.

24. The composition of embodiments 1-23 which has antimicrobial properties.

25. The composition of embodiments 1-24 wherein a weight percent of the substrate is greater than a weight percent of the silver particle.

26. The composition of embodiments 1-25 wherein a weight percent of the substrate is greater than 75% and a weight percent of the silver particles is less than 25%.

27. A method of preparing the composition of embodiments 1-26, the method comprising:
a. providing an aqueous composition comprising the silver particle;
b. combining the composition of (a) with the solid substrate to provide an aqueous silver composition; and
c. removing water from the aqueous silver composition of (b) to provide the anhydrous composition.

28. The method of embodiment 27 wherein (c) is accomplished through evaporation of the water.

29. The method of embodiment 27 wherein (c) is performed in a freezer dryer.

30. The method of embodiment 27 wherein (c) provides the composition of embodiment 1 in a powdered form.

31. A method of preparing an aqueous composition comprising combining an anhydrous composition of embodiments 1-26 with water.

32. A method of imparting antimicrobial properties to a substrate, the method comprising:
a. combining the substrate with a composition comprising silver particle and water to provide an aqueous composition; and
b. removing water from the aqueous composition to provide an anhydrous composition comprising the silver particle and the substrate.

33. The method of embodiment 32 wherein the substrate is selected from food and food additives, textiles, paper products and personal protective equipment, e.g., a mask.

34. A method for providing preserved food, the method comprising:
a. providing food, optionally providing solid food;
b. combining the food with an aqueous composition comprising silver particles to provide a precursor composition; and
c. removing water from the precursor composition to provide the preserved food.

35. The method of embodiment 34 comprising adding water to the preserved food.

All references disclosed herein, including patent references and non-patent references, are hereby incorporated by reference in their entirety as if each was incorporated individually.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. An anhydrous composition comprising a silver particle and a substrate.

2. The composition of claim 1 wherein the substrate is a synthetic sugar.

3. The composition of claim 2 wherein the synthetic sugar is selected from allulose, stevia, splenda, truvia, swerve, and purcane sweeteners.

4. The composition of claim 1 wherein the substrate is a natural sugar.

5. The composition of claim 4 wherein the natural sugar is a carbohydrate selected from cane sugar such as sucrose, beet sugar such as sucrose, dairy sugar such as lactose, and fruit sugar such as fructose.

6. The composition of claim 1 wherein the substrate is a non-carbohydrate sweetener.

7. The composition of claim 6 wherein the non-carbohydrate sweetener is selected from sorbitol and xylitol.

8. The composition of claim 1 wherein the substrate is an inorganic material selected from aluminosilicate, silicon oxide such as silicon dioxide including silica, and a metal oxide such as aluminum oxide, zinc oxide and calcium oxide.

9. The composition of claim 1 wherein the substrate is a polymer.

10. The composition of claim 9 wherein the polymer is a synthetic polymer.

11. The composition of claim 10 wherein the synthetic polymer is selected from polyethylene and polypropylene.

12. The composition of claim 1 wherein the substrate is a solid.

13. The composition of claim 1 wherein the composition is a solid.

14. The composition of claim 1 wherein the substrate is in a powdered form.

15. The composition of claim 1 wherein the substrate is fibrous.

16. The composition of claim 1 wherein the substrate is selected from food and food additives.

17. The composition of claim 1 wherein the substrate is a textile.

18. The composition of claim 1 wherein the substrate is a paper product.

19. The composition of claim 1 wherein the substrate is personal protective equipment, e.g., a mask.

20. The composition of claim 1 in a powdered form.

21. The composition of claim 1 wherein the silver particle is a coating on the substrate.

22. The composition of claim 1 wherein the silver particle is a coating of silver particles evenly distributed on a surface of the substrate.

23. The composition of claim 1 wherein the silver particle adheres to the substrate.

24. The composition of claim 1 which has antimicrobial properties.

25. The composition of claim 1 wherein a weight percent of the substrate is greater than a weight percent of the silver particle.

26. The composition of claim 1 wherein a weight percent of the substrate is greater than 75% and a weight percent of the silver particles is less than 25%.

27. A method of preparing the composition of claim 1, the method comprising:
a) providing an aqueous composition comprising the silver particle;
b) combining the composition of (a) with the solid substrate to provide an aqueous silver composition; and
c) removing water from the aqueous silver composition of (b) to provide the anhydrous composition.

28. The method of claim 27 wherein (c) is accomplished through evaporation of the water.

29. The method of claim 27 wherein (c) is performed in a freezer dryer.

30. The method of claim 27 wherein (c) provides the composition of claim 1 in a powdered form.

31. A method of preparing an aqueous composition comprising combining an anhydrous composition of claim 1 with water.

32. A method of imparting antimicrobial properties to a substrate, the method comprising:
a) combining the substrate with a composition comprising silver particle and water to provide an aqueous composition; and
b) removing water from the aqueous composition to provide an anhydrous composition comprising the silver particle and the substate.

33. The method of claim 32 wherein the substrate is selected from food and food additives, textiles, paper products and personal protective equipment, e.g., a mask.

34. A method for providing preserved food, the method comprising:
a) providing food, optionally providing solid food;
b) combining the food with an aqueous composition comprising silver particles to provide a precursor composition; and
c) removing water from the precursor composition to provide the preserved food.

35. The method of claim 34 comprising adding water to the preserved food.
